(19) United States
(12) Patent Application Publication
(10) Pub. No.: US 2013/0060096 A1
(43) Pub. Date: Mar. 7, 2013

(54) Integrating Wellness Care and Data into the Patient Care Cycle

(71) Applicants: Doreen Lynn Galli, Dripping Springs, TX (US); Marc S. Galli, Dripping Springs, TX (US); Darryl W. Jackson, Norcross, GA (US)

(72) Inventors: Doreen Lynn Galli, Dripping Springs, TX (US); Marc S. Galli, Dripping Springs, TX (US); Darryl W. Jackson, Norcross, GA (US)

(73) Assignee: PATIENT FORWARD, LLC, Dripping Springs, TX (US)

(21) Appl. No.: 13/226,517

(22) Filed: Sep. 7, 2011

(51) Int. Cl. A61B 5/00

(52) U.S. Cl. 600/300

(57) ABSTRACT

In a computer-based medical patient care system, patient wellness information, such as exercise data, visits to wellness providers, and vitamin intake, is collected by the system. The wellness information is associated with the patient=s medical record. The wellness information is made available to the patient=s the medical services providers via the system. Wellness information is categorized relative to whether it has been verified by a third party or self reported by the patient. Medical providers, such as physicians, obtain insight into patients=wellness issues, thereby becoming able to work with patients more effectively to effect behavior modification, determine whether the patient is actually following wellness directives, etc.

### BACKGROUND OF THE INVENTION

1. Field of the Invention

The present invention relates to capturing wellness information, task items and care information into an online medical environment for retention and use by patients, wellness providers and medical service providers. More particularly, the present invention relates to capturing patient information not normally captured and including this information in records and workflow associated with patients for use by medical service providers, wellness providers and patients.

2. Description of the Related Art

Traditionally, the maintenance of patient medical records has been the responsibility of the medical profession. This responsibility and burden has let the medical profession decide what should and should not be included in a patient=s medical records. Given what has been traditionally a huge trail of paper and the state of technology, the bias has traditionally been to include only materials from medical professionals that are easy to obtain. The patient is left out of the loop as to what should be included and the records are difficult for the patient to obtain. Further, as time passes, there are often multiple sets of an individual patient=s medical records, and these records often do not match or are incomplete.

Additionally, wellness providers, particularly those who may not qualify as medical professionals, and the services they provide, are also often left out of the loop. Medical records typically do not include significant wellness records, and usually lack feedback as to whether or not wellness issues or directives have actually been carried out by a patient.

Given this bias and the state of the technology, additional data which might be of great use to medical providers has traditionally not been included in a patient=s medical records, medical tools or medical systems.

A physician or other medical provider is limited by what patient information is available to them in terms of the wellness side of the patient equation. A medical provider is very limited as to her control of the patient when a patient is not in a controlled medical environment, such as a hospital. Medical tests and records provide a medical provider with part of the equation. But often a medical provider has to rely on a patient=s memory and truthfulness for another part of the equation. And given that a patient may be giving the medical provider wrong or incomplete information, intentionally or not, the result is often a missed diagnosis, unnecessary tests, unsuccessful preventative treatment, incomplete treatment, etc. The result can be medical conditions that should be controllable advancing to a more serious state, the need for more expensive treatment options (such as surgery), etc.

Accordingly, including the wellness side of the equation into a patient=s medical records can increase the accuracy of medical treatment, reduce costs, improve the condition of the patient, etc.

Some attempts have been made to address the wellness aspect of a patient=s overall medical picture. Insurance companies and companies have introduced programs to induce their customers and employees to live healthier lifestyles by offering incentives for exercise, quitting smoking, etc. But these wellness progress indicators are self reported and not made part of a patient=s medical record. Wellness providers are seldom included in the loop with physicians, and accurate feedback to physicians is limited. This lack of inclusion of wellness feedback limits the effectiveness of desired wellness activities. To increase the effectiveness and treatment, and to increase the chance of successful behavioral changes, a technique to include wellness providers and their patient information into the loop is desirous.

U.S. Pat. No. 7,912,737 to Schoenberg provides a discussion of patient wellness in connection with a telemedicine/medical services brokerage environment. The patent describes a consumer advisor functionality which is utilized to drive engagements between consumers (patients) and medical services providers. The consumer advisor functions to query a consumer and develop a set of recommendations to the consumer for potential medical engagements. One area in which the consumer advisor functions is described as wellness. But wellness in this patent is in terms of a patient=s current health and wellness, i.e., what is the patient=s current body mass index, has the patient had a recent mammogram, what is the patient=s cholesterol level, etc.

This patent fails to address the overall wellness of a patient beyond suggesting potential engagements with medical service providers who are part of the medical brokerage system. The concept of wellness also appears to be limited in scope to diagnosing potential areas of concern based on current medical data and what kinds of appointments or medical tests a patient should have based on age and the passage of time. The overall wellness of a patient in terms of exercise, proper use of prescriptions, intake of vitamins, proper and regular use of physical therapy, class attendance, etc., are not addressed. Further, the technique described in the patent does not incorporate wellness activities and feedback into the patient=s treatment, records, and doctor-patient communications.

Additionally, the medical services provider is not included in the information loop. No feedback is provided to the medical services provider. Providers of wellness services, such as pharmacists, physical therapists, exercise class teachers, etc., and the care they provide are not treated on an equal footing with physicians and physician-provided care in the prior art. The care provided by wellness service providers is often prescribed or ordered by physicians and is an essential part of the overall health or health maintenance or recovery of a patient. However, other than self reporting on a questionnaire or verbal reporting by a patient, this information is not captured and the accuracy is questionable. The lack of data integrity essentially eliminates the use of wellness data as an effective tool by physicians.

Accordingly, a need exists for a technique that integrates wellness providers into the whole picture of patient health, captures wellness data and integrates wellness data the health record of a patient for use by medical services providers.

### SUMMARY OF THE INVENTION

The present invention provides a technique for integrating wellness care and data into the patient care cycle.

In one aspect, addressed in the present application computer-implemented method for collecting patient wellness information and associating the collected patient wellness information with patient medical information and making the combined information available to a medical services provider in a computer-based medical-wellness system is provided. The method comprises receiving wellness information for a patient at the medical-wellness system and associating the received patient wellness information with a patient medical record, and providing the patient wellness information to a medical provider portal for the medical-wellness system when the medical services provider accesses the patient medical record for the patient from the medical provider portal and requests the patient wellness information.

Preferably, when the received patient wellness information is input to the medical-wellness system by a wellness provider, the wellness information is deemed to be provider-reported data, and the providing step further comprises associating an indication with the wellness data that the patient wellness information is provider-reported data. Further, when the received patient wellness information is input to the medical-wellness system by the patient, the wellness information is deemed to be patient-reported data, and the providing step further comprises associating an indication with the wellness information that the wellness information is patient-reported data.

Additionally, the wellness information is associated with wellness items, and the computer-implemented method further comprises the medical services provider creating a wellness item for a patient from a list of available wellness items provided by the medical portal. Also, the receiving step may further comprise receiving the wellness information from a patient portal to the medical-wellness system, the patient self-reporting the wellness information via the patient portal. Further, the receiving step may further comprise receiving the wellness information from a wellness provider portal to the medical-wellness system, the wellness provider inputting the patient information via the wellness portal.

In another aspect, techniques are provided for programmatically collecting patient wellness information and associating the collected patient wellness information with patient medical information and making the combined information available to a medical services provider in a medical-wellness environment. This aspect preferably comprises an apparatus comprising a processor and a computer program product residing on a computer readable medium, the computer program product comprising instructions for causing the processor to receive wellness information for a patient at the medical-wellness environment and associate the received patient wellness information with a patient medical record, and provide the patient wellness information to a medical provider portal for the medical-wellness environment when the medical services provider accesses the patient medical record for the patient from the medical provider portal and requests the patient wellness information.

In still another aspect, techniques are provided in a computer program product residing on a computer readable medium for collecting patient wellness information and associating the collected patient wellness information with patient medical information and making the combined information available to a medical services provider in a medical-wellness environment. The computer program product comprising instructions for causing a computer to receive wellness information for a patient and associate the received patient wellness information with a patient medical record, and provide the patient wellness information to a medical provider portal when the medical services provider accesses the patient medical record for the patient from the medical provider portal and requests the patient wellness information.

The present invention will now be described with reference to the following drawings, in which like reference numbers denote the same element throughout.

### BRIEF DESCRIPTION OF THE DRAWINGS

FIG. 1 provides a computing and networking environment in which the present invention may be practiced;

FIG. 2 illustrates a graphical user interface displayed at a medical provider portal to a medical services provider in accordance with the present invention;

FIG. 3 is a flow chart describing how the interactions of a wellness provider with a patient are captured and wellness information created for use by medical providers and others within the system according to the present invention;

FIG. 4 illustrates a graphical user interface displayed at a patient portal to a patient in accordance with the present invention; and

FIG. 5 is a flow chart describing the how patient wellness information can be entered into the medical-wellness environment according to the present invention.

### DESCRIPTION OF PREFERRED EMBODIMENTS

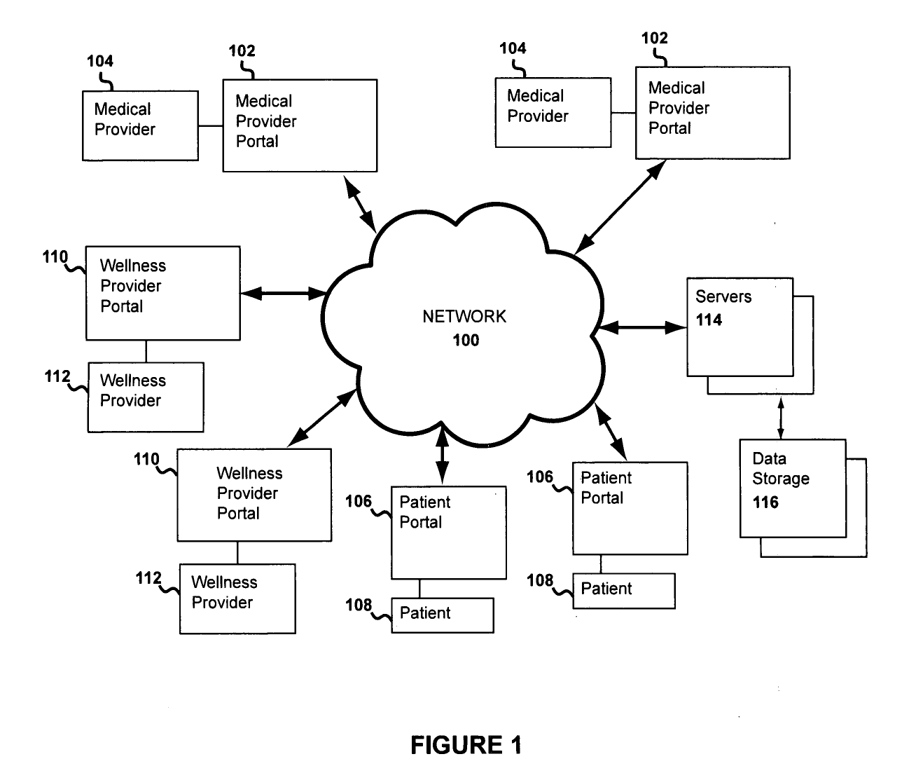

FIG. 1 illustrates a cloud-based network environment in which a medical-wellness software system according to the present invention may be practiced. Preferably, the medical-wellness software system utilizes the cloud computing concept, in that the system operates as a service to users and is accessed via the Internet. Alternatively, in some implementations, the network may be a local Intranet. In either case, a network 100 is utilized to enable communication between medical provider portals 102 to be utilized by medical service providers 104, patient portals 106 to be utilized by patients 108, wellness provider portals 110 to be utilized by wellness providers 112, and one or more servers 114. The servers 114 have data storage devices 116 associated therewith. The medical service providers 104, patients 108 and wellness providers 112 utilize web browsers at their point of access to the network 100 to access their respective portals 102, 106, 110, which are maintained by the servers 114 as part of a cloud-based medical-wellness software service. The point of access can be one of many devices, including a laptop computer, personal computer, kiosk dedicated to providing a connection to the medical software, tablet, smart phone, or any device capable of an internet connection and browsing the Internet. A web cam associated with the device to enable a video link is preferred, as the medical-wellness system enables a two way video link between users of the system in accordance with known techniques. While most devices do not require any software other than a standard web browser to be installed to work with the medical-wellness system, some small devices having web browsers with limited functionality may require a local support application to be installed to enable some of the features of the system.

The portals 102, 106, 110 provide their respective users with tools via graphical user interfaces (GUIs) by which the users interact with the medical-wellness software system. Accordingly, a graphical user interface displayed for a medical services provider when a medical services provider accesses her portal 102 via a web browser will be different than the GUI displayed for a patient via the patient portal 106 or the GUI for a wellness provider displayed for a wellness provider via the wellness provider portal 110.

The patient portal 106 provides patients with a single point of access at which all of their medical information can be maintained and accessed by the patients. In this way, a patient can have access to doctors names and contact information, current and historical appointment data, current and historical prescription information, wellness information, insurance information, x-rays, allergy information. In other words, a patient can have her complete medical history easily accessible, together with pertinent current medical/physician/prescription information.

The patient data accessible by wellness providers is limited by patient confidentiality laws and regulations and corresponding permissions granted by the patient. The wellness provider portal 110 enables the wellness provider to receive allowable or allowed information regarding a patient and create and input wellness information regarding the patient based on the patient-wellness provider interaction. When this wellness information is input into the medical-wellness system, it becomes part of a patient wellness record and is associated with the patient medical file, in accordance with known techniques. This wellness information will then be available to both patients and the patients=medical services providers via their respective portals

The concept of wellness providers in accordance with the present invention includes a variety of professionals and service providers, including physical therapists, dieticians, personal trainers, tai chi instructors, yoga instructors, pharmacists, etc. Using the telemedicine aspect of the medical-wellness software system according to the present invention, wellness providers may interact or conduct appointments with a patient. Additionally, the medical-wellness system preferably includes recorded wellness-related classes and wellness information, which are stored on the data storage devices 116 associated with the servers 114. For example, a series of aerobics classes, tai chi classes, yoga classes, etc. can be stored so that a patient can attend and participate in a wellness class from home or other location at his convenience. The video aspect of the medical-wellness system permits the patient to view the class via his device which accesses the patient portal 106. The fact that a patient accessed a class is recorded within the medical-wellness system and added to the patient=s wellness record maintained by the medical-wellness system.

According to one aspect of the invention, a wellness provider may be a physical therapist. For example, for a patient with a shoulder injury, a physician refers the patient to the physical therapist for treatment. The referral is recorded in the medical-wellness software system by the physician or the physician=s staff via the medical provider portal 102, and becomes part of the patient=s medical record. So the fact that a referral to a physical therapist was made will be available to the medical service provider via the medical provider portal 102 when the medical provider accesses the patient=s record.

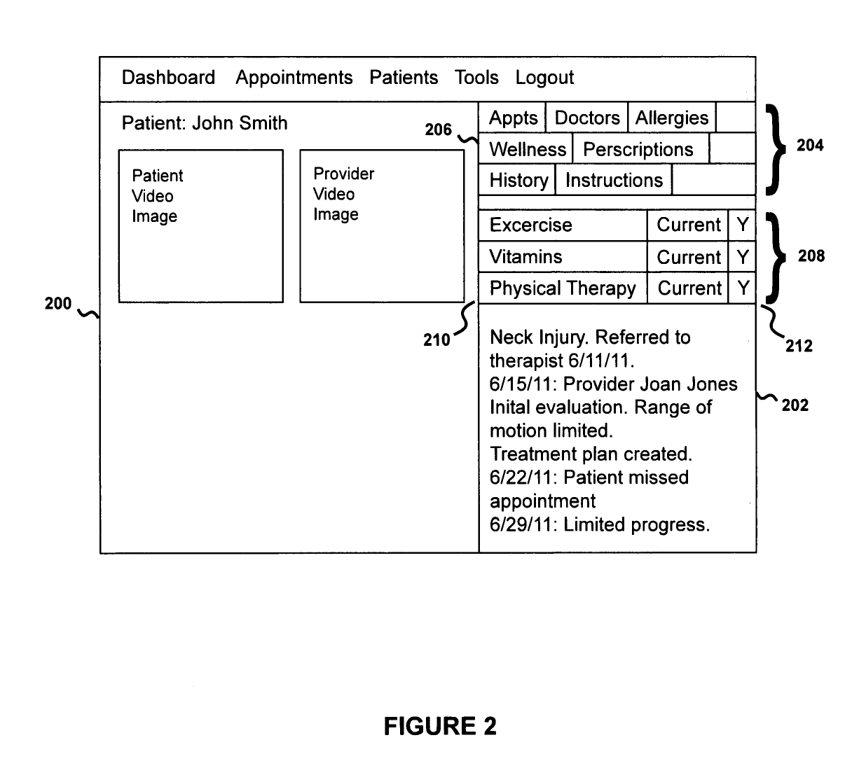

FIG. 2 illustrates one screen of a medical services provider graphical user interface (GUI) 200, which is displayed to a medical services provider via the medical provider portal 102. The GUI 200 includes an information window 202 associated with a set of tabs 204. The information window 202 and tabs 204 will display patient information for a patient whose file the medical provider has opened. The patient data is retrieved from the data storage devices 116. A wellness tab 206, when selected, causes patient wellness information to be displayed in the information window 202 within the GUI 200. Preferably, a list of wellness issues 208 and their current status are listed within the window 202. The wellness list 208 may include recommended tests or health habits based on the patient=s age, gender, lifestyle, etc., as previously identified by a medical services provider or determined automatically by the medical-wellness system based on current recommendations by the American Medical Association, government health organizations, etc. For example, current recommendations for a 50 year old male may call for 30 minutes of exercise four times a week and a colonoscopy. Each wellness item 210 in the wellness list 208 is selectable, in that its selection opens in the information window 202 details about the wellness item.

Accordingly, when the medical services provider refers a patient to a physical therapist, an entry is made into the medical-wellness system, and the wellness list is updated to include the referral as a wellness item. A selected physical therapist or physical therapy service then receives the referral and sets an appointment with the patient via the medical-wellness software system or via more traditional techniques. In either case, the wellness provider portal 110 may be utilized in a number of ways relative to the physical therapy. First, a traditional in-person appointment can be conducted. However, if an in-person appointment is not needed, the appointment can be conducted utilizing the two-way video aspect of the medical-wellness software system, in accordance with known techniques. In this way, a live appointment is conducted utilizing the video feature. In either case, the wellness provider is added to the patient=s provider list maintained on the medical-wellness system, either by the physician or the physician=s delegate or by the patient, utilizing their respective portal. Adding the wellness provider to the patient=s network of providers gives the wellness provider access to permitted patient information maintained on the medical-wellness software system. The patient=s name will be added to the patient list for the wellness provider in the medical-wellness system and can be accessed via the wellness provider portal 110.

Similarly, the physical therapist is added to a provider list for the patient in the medical-wellness system. Accordingly, name of the physical therapist and related information will be accessible by the patient via the patient portal 106.

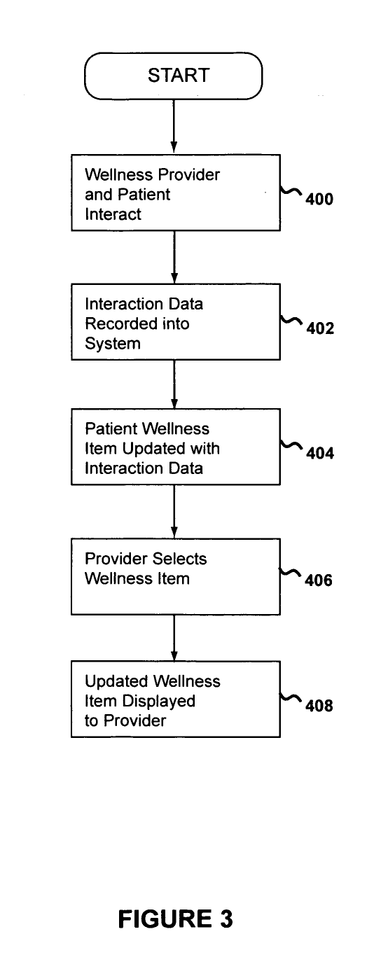

FIG. 3 is a flowchart which illustrates the process by which a wellness provider provides wellness information into the medical-wellness system for use by other users of the system. After the patient and the physical therapist have conducted their appointment, either in person or via the video feature (Step 400), the fact the appointment was conducted, the treatment plan, and comments of the physical therapist (wellness provider-patient interaction) are entered into the medical-wellness software system (Step 402) via the wellness provider portal 110. The medical-wellness system then updates the wellness item with this information (Step 404), and the information becomes available to medical service providers via their medical provider portal 102. Subsequently, a medical services provider can access the updated wellness for the wellness item information via the medical provider portal 102 by selecting the patient from a patient list, selecting the wellness tab 206, and selecting the appropriate wellness item 210 from the list 208 of wellness items in the information window 202 (Step 406). For example, this wellness item may be entitled Aphysical therapy@ and include appropriate identifying information, such as the date of the referral and issue being addressed, so that the medical provider can quickly and easily identify the desired wellness item. By selecting this wellness item, the record of any appointments between the patient and the physical therapist will be displayed, along with any appointment notes entered by the physical therapist (Step 408). Notes can include the regimen prescribed by the physical therapist, whether the patient is improving over time, whether the patient appears to be performing the assigned exercises on his own time, etc. The information is indicated as having been entered by the wellness provider, as opposed to being entered or reported by the patient. This indicator provides the medical services provider with a greater level of trust in the information, since the medical-wellness system has indicated it was entered by a wellness provider and not by the patient. Accordingly, an indicator 212 is associated with wellness entry information indicate who entered the information into the medical-wellness system.

In this way, the medical services provider is provider with an easy way to know with a high degree of accuracy whether the patient has been seeing the physical therapist and the progress or lack of progress being made.

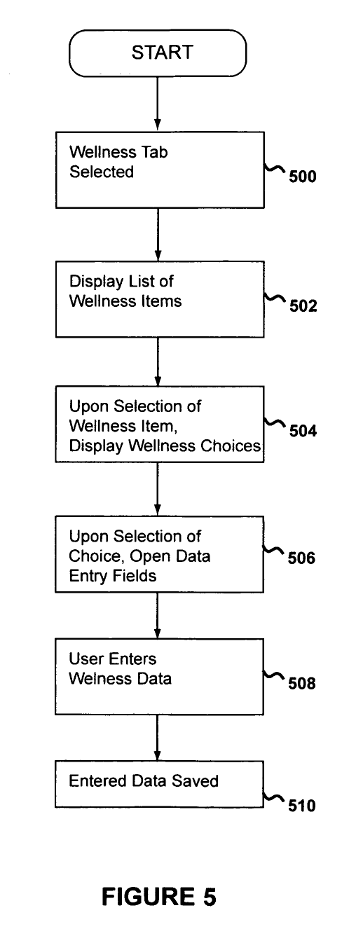

The wellness reporting technique of the present invention can also be utilized to provide the medical services provider of different types of wellness information and to assist the patient in keeping track of his own wellness data. For example, a medical services provider may recommend that the patient participate in 30 minutes of rigorous exercise at least four times a week. The medical-wellness system provides the patient with a number of options in maintaining and reporting this kind of wellness information. The patient can exercise independently, keep track of his efforts, and then enter his exercise history into the medical-wellness software system himself. The self-reporting process will now be described with reference to the flowchart of FIG. 5.

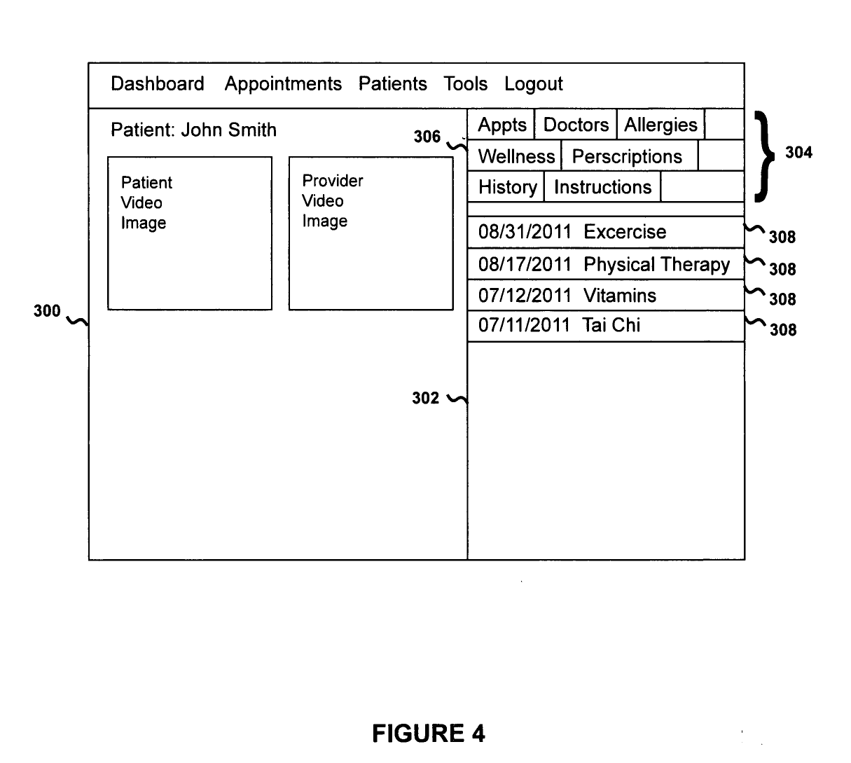

FIG. 4 illustrates one screen of a patient graphical user interface (GUI) 300, which is displayed to the patient via the patient portal 106. To self report exercise information, the patient uses his patient portal 106 to access the patient graphical user interface 300 and selects a wellness tab 306 (Step 500) from a set of tabs 304 associated with an information window 302. The medical-wellness system displays a list of selectable wellness items 308 in the information window 302, such as an exercise option (Step 502). The wellness items 308 preferably include exercise, vitamin use tracking, prescription medicine use tracking, attendance of health-related classes, etc. As per Step 504, selection of one of the wellness items 308 can cause a subset of wellness choices under the item to be displayed, or may simply permit the patient to enter information related to his wellness efforts for that wellness item for a selected date. For example, when a patient selects an exercise option from the list of wellness items 308, a series of exercise sub-options are displayed (Step 506), such as running, walking, biking, swimming, rowing, in-line skating, cross country skiing, use of elliptical machines, etc. Selection of one of the sub-options causes it to be added to an active exercise list. For a specific exercise, the patient would enter or select via an available calendar the date of the exercise, the duration of the exercise and any patient notes regarding the exercise (Step 508). Since the patient self-reported this information, an indication that the information was self-reported is included with the data. The self-reported indication would be displayed to any medical services provider or wellness provider who reviews this information. The entered wellness data is saved in the medical-wellness system in accordance with known techniques (Step 510) and is subsequently accessible by any authorized medical services provider or wellness provider.

Similarly, an instructor/leaser of a spin (cycling) class or exercise group can be considered a wellness provider and, if authorized, enter exercise information into the medical-wellness system for the patient using the wellness provider portal 110 and the process described above relative to entering patient wellness information for a patient. This information will be considered by the system to be wellness provider reported. Accordingly, when a medical services provider or wellness provider subsequently reviews wellness this information for the patient, an indication that the information was wellness provider reported will appear with the information. This indication gives the reviewer a higher level of confidence that the information is accurate.

Prescription medicine and pharmacies may also be considered to be part of the wellness cycle. Accordingly, wellness information may include information about the patient=s usage of prescriptions. For example, when a medical services provider prescribes a drug to the patient, the date that prescription was actually filled (if it was filled at all) can be entered by the pharmacy that filled the prescription or the insurance provider which paid for part of the prescription. Both the pharmacy and the insurance company can be considered to be wellness providers. As described above with respect to other wellness providers, an employee associated with the pharmacy or insurance company would enter into the medical-wellness system that the prescription was filled. Alternatively, the pharmacy or insurance company may be linked to the medical-wellness system. The filling of the prescription by the pharmacy could then be automatically sent to the medical-wellness system.

The benefit of having a pharmacy as part of the process would indicate to the medical services provider that a prescription had actually been filled. Once again, this information would be accessed by the medical services provider via the medical provider portal 102. The prescription information may be viewed via the wellness tab 206 and/or a prescription tab 214, and can provide the medical services provider a more complete picture of the patient=s health and recovery, and whether the patient is following medical instructions. This information can be used in conjunction with any self-reporting of the usage of the prescription information by the patient through the wellness self-reporting process.

Of further use might be an indicator of when and if a prescription was refilled. If the meditation was supposed to be taken over a lengthy period of time and refills were required, information on the date (if any) that a prescription was refilled could be invaluable to a prescribing medical services provider. If a medical services provider prescribes a 30 day supply of a medication, with one of more refills, and the prescription is not refilled at all or refilled more than 30 days after the original prescription was filled, the medical services provider will have a good indication that the patient is not taking the medication as prescribed.

As will be appreciated by one of skill in the art, embodiments of the present invention may be provided as (for example) methods, systems, and/or computer program products. The present invention may take the form of an embodiment, or an embodiment combining software and hardware aspects. Furthermore, the present invention may take the form of a computer program product which is embodied on one or more computer-usable storage media (including, but not limited to, disk storage, CD-ROM, optical storage, and so forth) having computer-usable program code embodied therein.

The present invention has been described with reference to flow diagrams and/or block diagrams according to embodiments of the invention. It will be understood that each flow and/or block of the flow diagrams and/or block diagrams, and combinations of flows and/or blocks in the flow diagrams and/or block diagrams, can be implemented by computer program instructions. These computer program instructions may be provided to a processor of a general purpose computer, special purpose computer, embedded processor, or other programmable data processing apparatus to produce a machine, such that the instructions, which execute the more processor of the computer or other programmable data processing apparatus, create means for implementing the functions specified in the flow diagram flow or flows and/or block diagram block or blocks.

These computer program instructions may also be stored in a computer-readable memory that can direct a computer or other programmable data processing apparatus to function in a particular manner, such that the instructions stored in the computer-readable memory produce an article of manufacture including instruction means which implement the function specified in the flow diagram flow or flows and/or block diagram block or blocks.

The computer program instructions may also be loaded onto a computer or other programmable data processing apparatus to cause a series of operational steps to be performed on the computer or other programmable apparatus to produce a computer implemented process such that the instructions which execute on the computer or other programmable apparatus provide steps for implementing the functions specified in the flow diagram flow or flows and/or block diagram block or blocks.

While preferred embodiments of the present invention have been described, additional variations and modifications in those embodiments may occur to those skilled in the art once they learn of the basic inventive concepts. Therefore, it is intended that the appended claims shall be construed to include preferred embodiments and all such variations and modifications as fall within the spirit and scope of the invention.

1. A computer-implemented method for collecting patient wellness information and associating the collected patient wellness information with patient medical information and making the combined information available to a medical services provider in a computer-based medical-wellness system, comprising:
receiving wellness information for a patient at the medical-wellness system and associating the received patient wellness information with a patient medical record; and
providing the patient wellness information to a medical provider portal for the medical-wellness system when the medical services provider accesses the patient medical record for the patient from the medical provider portal and requests the patient wellness information.

2. The computer-implemented method according to claim 1, wherein when the received patient wellness information is input to the medical-wellness system by a wellness provider, the wellness information is deemed to be provider-reported data, said providing further comprises associating an indication with the wellness data that the patient wellness information is provider-reported data.

3. The computer-implemented method according to claim 1, wherein when the received patient wellness information is input to the medical-wellness system by the patient, the wellness information is deemed to be patient-reported data, and said providing further comprises associating an indication with the wellness information that the wellness information is patient-reported data.

4. The computer-implemented method according to claim 1, wherein the wellness information is associated with wellness items, said computer-implemented method further comprising the medical services provider creating a wellness item for a patient from a list of available wellness items provided by the medical portal.

5. The computer-implemented method according to claim 1, wherein said receiving step further comprise receiving the wellness information from a patient portal to the medical-wellness system, the patient self-reporting the wellness information via the patient portal.

6. The computer-implemented method according to claim 1, wherein said receiving step further comprises receiving the wellness information from a wellness provider portal to the medical-wellness system, the wellness provider inputting the patient information via the wellness portal.

7. An apparatus comprising:
a processor; and
a computer program product residing on a computer readable medium for collecting patient wellness information and associating the collected patient wellness information with patient medical information and making the combined information available to a medical services provider in a medical-wellness environment, the computer program product comprising instructions for causing the processor to:
receive wellness information for a patient at the medical-wellness environment and associate the received patient wellness information with a patient medical record; and
provide the patient wellness information to a medical provider portal for the medical-wellness environment when the medical services provider accesses the patient medical record for the patient from the medical provider portal and requests the patient wellness information.

8. A computer program product residing on a computer readable medium for collecting patient wellness information and associating the collected patient wellness information with patient medical information and making the combined information available to a medical services provider in a medical-wellness environment, the computer program product comprising instructions for causing a computer to:
receive wellness information for a patient and associate the received patient wellness information with a patient medical record; and
provide the patient wellness information to a medical provider portal when the medical services provider accesses the patient medical record for the patient from the medical provider portal and requests the patient wellness information.
